(19) United States
(12) Patent Application Publication
(10) Pub. No.: US 2016/0151044 A1
(43) Pub. Date: Jun. 2, 2016

(54) ULTRASONIC PROBE

(71) Applicant: SAMSUNG ELECTRONICS CO., LTD., Suwon-si (KR)

(72) Inventors: Youngil KIM, Suwon-si (KR); Jong Keun SONG, Yongin-si (KR); Eunsung LEE, Hwaseong-si (KR); Kyungil CHO, Seoul (KR); Minseog CHOI, Seoul (KR)

(73) Assignee: SAMSUNG ELECTRONICS CO., LTD., Suwon-si (KR)

(21) Appl. No.: 14/886,641

(22) Filed: Oct. 19, 2015

(30) Foreign Application Priority Data
Dec. 1, 2014 (KR) 10-2014-0169983

(51) Int. Cl. A61B 8/00

(52) U.S. Cl. CPC A61B 8/4455; A61B 8/429

(57) ABSTRACT

A ultrasonic probe including: a case including: an opening; and a coupling portion; and a probe lens comprising: a first portion being exposed through the opening and a second portion coupled to the coupling portion of the case, wherein one of the coupling portion of the case and the second portion of the probe lens comprises a groove, and other one of the coupling portion of the case and the second portion of the probe lens comprises a shape matching the groove to couple the case and the probe lens to each other.

### CROSS-REFERENCE TO RELATED APPLICATION

This application claims priority from the Korean Patent Application No. 10-2014-0169983, filed on Dec. 1, 2014, in the Korean Intellectual Property Office, the disclosure of which is incorporated herein by reference in its entirety.

### BACKGROUND

1. Field

Apparatuses consistent with exemplary embodiments relate to an ultrasonic probe to obtain ultrasonic images.

2. Description of the Related Art

An ultrasonic imaging apparatus is an apparatus configured to transmit ultrasonic signals toward a target portion located in an inside a body from a surface of the body, and non-invasively obtain images related to cross sections or blood flow of a soft tissue by using information from the reflected ultrasonic signals, that is, ultrasonic echo signals.

The ultrasonic image apparatus, when compared to other image diagnostic apparatus such as an x-ray diagnostic apparatus, an x-ray CT (Computerized Tomography) scanner, a MRI (magnetic Resonance Image), and a nuclear medicine diagnostic apparatus, is smaller in size, is less expensive, has better capabilities of displaying information on real-time basis, and is provided to be safer with respect to exposure to radiation. Therefore, the ultrasonic image apparatus is widely used for diagnoses of hearts, abdomens, and urinary system, and in obstetrics.

### SUMMARY

One or more exemplary embodiments provide an ultrasonic probe capable of preventing ultrasonic gel or cleansing solution from being introduced into an inside the ultrasonic probe at the time of ultrasonic test. In addition, One or more exemplary embodiments provide a probe lens of the ultrasonic probe which may be prevented from abrasion by outside pressure and friction.

In accordance with an aspect of an exemplary embodiment, there is provided an ultrasonic probe includes a case and a probe lens. The case may be provided with an opening formed thereto. The probe lens may have at least a portion thereof exposed through the opening, and one side thereof coupled to the case. At the coupling portion of the case and the probe lens, a groove may be formed on one of the case and the probe lens, while the other one of the case and the probe lens may be provided to have a shape corresponding to the groove and is coupled to the one of the case and the probe lens.

The case may be provided with a protrusion protruded toward a front.

The protrusion may be injection-molded integrally with the probe lens.

The protrusion may be injection-molded integrally with the case.

The protrusion may be positioned at an outer side of the opening.

The protrusion may be provided in a plurality of units thereof while spaced apart to each other.

The protrusion may be provided in the shape of a semi-sphere.

The protrusion may be provided in the shape of a polyhedron.

The probe lens may be injection-molded as the case is inserted.

The case may include a first surface positioned at a front, and a second surface bent at and extended from the first surface, and the groove may be formed at an inner side surface of the first surface or at an inner side surface of the second surface.

The probe lens may include a first portion exposed through the opening, a second portion extended from the first portion and coupled to an inner side surface of the case.

A front of the first surface may be provided with a plurality of protrusions formed thereto.

The groove may be provided by the shape of a mold at the time of the injection molding of the case as to be formed at an inner side surface of the case.

The groove may be provided by carving after the injection molding of the case.

In accordance with an aspect of an exemplary embodiment, there is provided an ultrasonic probe having a case provided with an opening formed thereto and a probe lens exposed through the opening, a plurality of grooves are formed on at least one portion of a rear surface of the case, and one side of the probe lens is mounted at a rear surface of the case while provided to correspond to the shape of the grooves, and a plurality of protrusions provided to make contact with an object may be provided at a front surface of the case.

The groove may be formed at an entire area of a rear surface of the case coupled to the probe lens.

The protrusion may be injection-molded integrally with the probe lens.

The protrusion may be provided at an outer side of the opening without covering the opening.

A moving path may be provided in between protrusions adjacent to each other among the plurality of protrusions so that ultrasonic gel is moved through the moving path.

The protrusion may be injection-molded integrally with the case

As is apparent from the above, an abrasion of a transducer or a printed circuit board positioned at an inside an ultrasonic probe by ultrasonic gel G or cleansing solution introduced in between a probe lens and a case at the time of ultrasonic test can be prevented by forming a concave-convex structure at an inner side surface of the case at which the probe lens is mounted.

In addition, lubricativeness can be improved and the pressure applied at the probe lens can be reduced as a certain amount of the ultrasonic gel is retained in between the probe lens and an object by the protrusion structure of a front of the ultrasonic probe.

In accordance with an aspect of an exemplary embodiment, there is provided an ultrasonic probe, including: a case including: an opening; and a coupling portion; and a probe lens including: a first portion being exposed through the opening and a second portion coupled to the coupling portion of the case, wherein one of the coupling portion of the case and the second portion of the probe lens comprises a groove, and other one of the coupling portion of the case and the second portion of the probe lens comprises a shape matching the groove to couple the case and the probe lens to each other.

The case may include a protrusion which protrudes from a front surface of the case.

The protrusion may be integrally formed with the probe lens.

The protrusion may be integrally formed with the case.

The protrusion may be provided at an outer side of the case with respect to the opening.

The protrusion may be one of a plurality of protrusions which are spaced apart with respect to one another.

The protrusion may include a cross-section shape of a semi-sphere.

The protrusion may include a cross-sectional shape of a polyhedron.

The probe lens may include an injection-molded lens.

The case may include: a first case portion positioned at a front side of the ultrasonic probe; and a second case portion bent at and extended from the first case portion, and wherein the groove is formed at an inner side surface of the first case portion or at an inner side surface of the second case portion.

The second portion may extend from the first portion and coupled to an inner side surface of the case.

A first surface of the first case portion may include a plurality of protrusions.

A shape of the at least one groove may correspond to a shape of a mold used for injection molding of the case.

The at least one groove may include a carved groove.

In accordance with an aspect of an exemplary embodiment, there is provided an ultrasonic probe including: a case provided with an opening; and a probe lens exposed through the opening, wherein: the case includes a plurality of grooves provided on at least one portion of a rear surface of the case, and the probe lens includes a shape corresponding to the plurality of grooves and is configured to be mounted at the rear surface of the case, and the case further includes a plurality of protrusions provided at a front surface of the case and configured to make contact with an object.

The groove may be provided over an entire area of the rear surface of the case coupled to the probe lens.

The protrusion may be provided at an outer side of the case with respect to the opening and is formed to be spaced apart from the opening.

A moving path may be provided between adjacent protrusions of the protrusions and configured to allow ultrasonic gel to move through the moving path.

### BRIEF DESCRIPTION OF THE DRAWINGS

The above and/or other aspects of the disclosure will become apparent and more readily appreciated from the following description of exemplary embodiments, taken in conjunction with the accompanying drawings of which:

FIG. 1 is a drawing illustrating an ultrasonic imaging apparatus in accordance with an exemplary embodiment.

FIG. 2 is a drawing illustrating an ultrasonic probe in accordance with an exemplary embodiment.

FIG. 3 is a cross-sectional view illustrating a case of an ultrasonic probe in accordance with an exemplary embodiment.

FIG. 4 is a cross-sectional view illustrating a case and a probe lens of an ultrasonic probe coupled to each other in accordance with an exemplary embodiment.

FIGS. 5A, 5B, 5C, 5D, and 5E are drawings illustrating various examples with respect to a shape of an inner side surface of an ultrasonic probe in accordance with an exemplary embodiment.

FIG. 6 is a cross-sectional view illustrating an ultrasonic probe in accordance with an exemplary embodiment.

FIG. 7 is a drawing illustrating a portion of an ultrasonic probe in accordance with an exemplary embodiment.

FIGS. 8A, 8B, 8C, 8D, and 8E are drawings illustrating various examples with respect to a shape of a case and a probe lens of an ultrasonic probe in accordance with an exemplary embodiment.

FIG. 9A and FIG. 9B are drawings illustrating a surface of an ultrasonic probe in accordance with an exemplary embodiment.

### DETAILED DESCRIPTION

Reference will now be made in detail to exemplary embodiments of the disclosure, examples of which are illustrated in the accompanying drawings, wherein like reference numerals refer to like elements throughout.

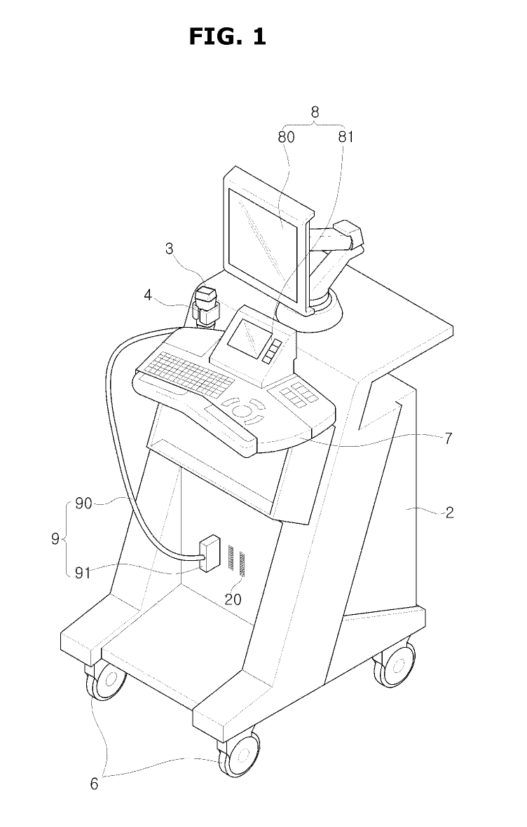

FIG. 1 is a drawing illustrating an ultrasonic imaging apparatus 1 in accordance with an exemplary embodiment.

Referring to FIG. 1, an ultrasonic imaging apparatus 1 in accordance with an exemplary embodiment includes a body 2, an ultrasonic probe 3, an input unit 7, and a display 8. The display 8 may include a main display 80 and a sub display 81.

The display 8 may display ultrasonic images obtained in ultrasonic diagnostic process. In addition, the display 8 may display applications related to motions of the ultrasonic imaging apparatus 1. In the exemplary embodiment, the main display 80 may display ultrasonic images obtained from an ultrasonic diagnostic process. The sub display 81 may display aspects related to motions of the ultrasonic imaging apparatus 1.

The main display 80 or the sub display 81 may be implemented in the form of a Cathode Ray Tube (CRT) or a Liquid Crystal Display (LCD). The main display 80 or the sub display 81 may be provided while coupled to the body 2, or may be provided as separate units separated from the body 2.

The body 2 may be provided with the input unit 7. The input unit 7 may be provided in the form of a keyboard, a foot switch, or a foot pedal. In a case when the input unit 7 is provided in the form of the keyboard, the input unit 7 may be provided at an upper portion of the body 2. In a case when the input unit 7 is provided in the form of the foot switch or the foot pedal, the input unit 7 may be provided at a lower portion of the body 2. A test administrator may control motions of the ultrasonic imaging apparatus 1 through the input unit 7.

The ultrasonic probe 3 may be connected to a connecting member 9. The connecting member 9 includes a cable 90 and a connector 91. One side of the cable 90 may be provided with the ultrasonic probe 3 and the other side of the cable 90 may be provided with the connector 91. As described above, the ultrasonic probe 3 and the body 2 may be connected to each other via the connecting member 9.

The ultrasonic probe 3 may be held at the body 2 via a holder 4 to be used by a test administrator. A test administrator may store the ultrasonic probe 3 by placing the ultrasonic probe 3 in the holder 4 when not using the ultrasonic probe 3.

The body 2 may be provided with a moving apparatus 6 to move the ultrasonic imaging apparatus 1. The moving apparatus 6 may be provided in the form of a plurality of casters provided at a lower surface of the body 2. The casters may be aligned as to drive the body 2 in a particular direction while provided to be freely moved, or may be locked as to stop at a particular position.

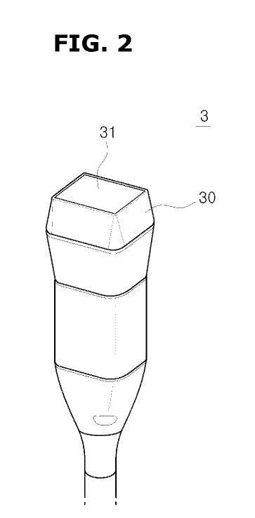

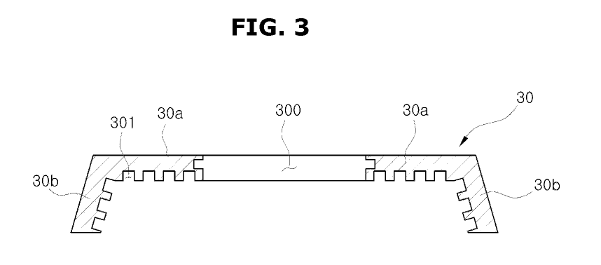

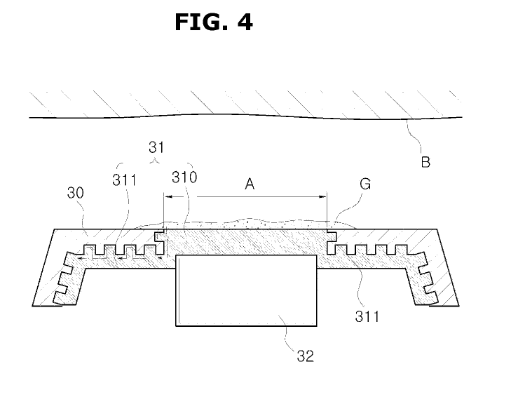

FIG. 2 is a drawing illustrating an ultrasonic probe 3 in accordance with an exemplary embodiment. FIG. 3 is a cross-sectional view illustrating a case 30 of an ultrasonic probe 3 in accordance with an exemplary embodiment. FIG. 4 is a cross-sectional view illustrating a case 30 and a probe lens 31 of the ultrasonic probe 3 coupled to each other in accordance with an exemplary embodiment.

Referring to FIGS. 2 and 3, the ultrasonic probe 3 in accordance with an exemplary embodiment includes a case 30 and a probe lens 31. An opening 300 is formed at a front of the case 30, and the probe lens 31 may be provided to make contact with a test body while exposed through the opening 300 of the case 30. An imaging with respect to the test body may be taken place by ultrasonic signals passing at a first portion 310 exposed through the opening 300 of the probe lens 31. The probe lens 31 may include a second portion 311 coupled to an inner side surface of a coupling portion 30c of the case 30.

A transducer 32, which generates ultrasonic signals, may be provided at an inside the case 30. The transducer 32 may be provided at a rear of the probe lens 31. The transducer 32 may include an acoustic matching layer (not shown), piezoelectric material (not shown), and an acoustic absorption layer (not shown). The acoustic matching layer, the piezoelectric material, and the acoustic absorption layer may be arranged at a rear of the probe lens 31 in the order in which the acoustic matching layer, the piezoelectric material, and the acoustic absorption layer are described.

The piezoelectric material is provided to perform a role release electric signals into air by converting the electric signals into ultrasonic signals, that is, acoustic signals, and convert the ultrasonic signals reflected from the air again into the electric signals.

The piezoelectric material may be composed of piezoelectric substance to generate ultrasonic signals by receiving electric signals and converting the electric signals into mechanical vibration. An effect in which a voltage is generated when a mechanical pressure is applied at predetermined substance and a mechanical transformation is taken place when the voltage is applied is referred to a piezoelectric effect or an inverse piezoelectric effect, and the substance having the effect as such is referred to as the piezoelectric substance. That is, the piezoelectric substance is referred to as the substance to convert electric energy into mechanical vibration energy and mechanical vibration energy into electric energy.

The acoustic matching layer may be positioned at a front of the piezoelectric material. The acoustic matching layer may perform a role to efficiently deliver the ultrasonic wave signals generated at the piezoelectric material to a test body by matching the acoustic impedance of the piezoelectric material and the acoustic impedance of the test body.

A protective layer may also be provided at a front of the acoustic matching layer. The protective layer is provided as to prevent an outside leakage of high frequency elements that may be generated at the piezoelectric material, and to block an introduction of outside high frequency signals to an inside.

The acoustic absorption layer may be disposed at a rear of the piezoelectric material. The acoustic absorption layer may reduce the pulse width of ultrasonic signals by restraining free vibration of the piezoelectric material, and prevent image distortion by blocking ultrasonic signals from being unnecessarily delivered toward a rear of the piezoelectric material.

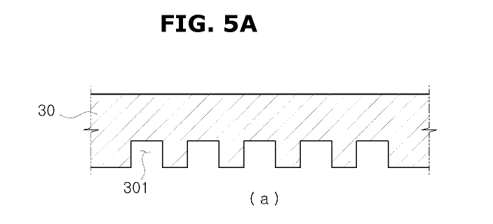

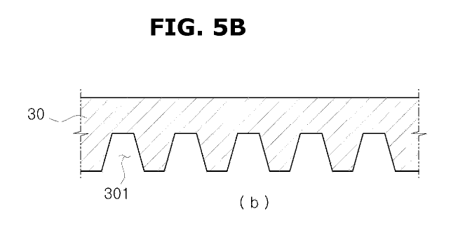

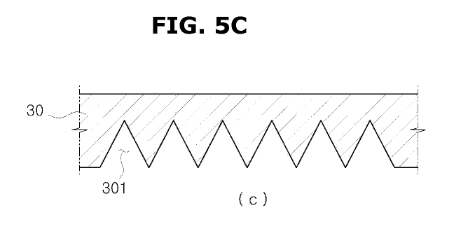

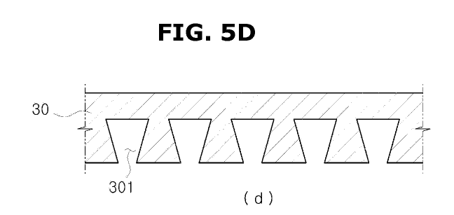

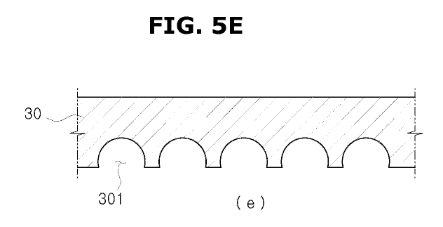

FIG. 5A to FIG. 5E are drawings illustrating various examples with respect to a shape of an inner side surface of the ultrasonic probe in accordance with one embodiment of the present disclosure.

Referring to FIG. 3, FIG. 4 and FIG. 5A to FIG. 5E, the ultrasonic probe 3 in accordance with an exemplary embodiment may be provided such that at least a portion of a surface of the case 30 and a portion of the probe lens 31 are overlapped with respect to each other. In an exemplary embodiment, at least a portion of a rear surface of the case 30 and a portion of a front surface of the probe lens 31 may be coupled to each other as to be overlapped with respect to each other.

Referring to FIG. 3, a groove 301 may be formed at the rear surface of the case 30. The case 30 may include a first case portion 30a provided with the opening 300 formed thereto, and a second case portion 30b bent at and extended from one end of the first case portion 30a. The groove 301 may be formed at least at one of a rear surface 30r of the first case portion 30a or at a rear surface of the second case portion 30b.

A front surface of the probe lens 31, which is overlapped with a surface of the case 30 provided with the groove 301 formed at the rear surface 30r thereof, may be provided with a shape thereof to match the shape of the groove 301. In an exemplary embodiment, the probe lens 31 may be injection-molded as the case 30 is inserted in a mold for injection molding. The injection material provided to become the probe lens 31, as the injection material is cooled after injected at an inside the groove 301 formed at the rear surface 30r of the case 30, may be injection-molded so that the probe lend 31 is provided with the shape corresponding to the groove 301 of the case 30.

The groove 301 may be formed by the shape of a mold at the time of the injection molding of the case 30. The groove 301 may be formed by carving the rear surface 30r of the case 30 after the injection molding of the case 30. The groove 301 may be formed at a portion of the rear surface 30r of the case 30 or at an entirety of the rear surface 30r of the case 30 in a regular pattern of in an irregular pattern.

The probe lens 31 may be injection-molded as the case 30 is inserted at the mold at the time of injection-molding the probe lens 31. The probe lens 31 may be provided to cover the portion of the opening 300 of the case 30, and the probe lens 31 may also be provided to cover at least a portion of the rear surface 30r of the case 30. One surface of the probe lens 31 exposed through the first case portion 30a and the opening 301 of the case 30 may form a coplanar surface with respect to a front surface of the first case portion 30a.

In an exemplary embodiment, the probe lens 31 may be injection-molded in the shape which corresponds to the groove 301 formed at an inner side surface of the case 30. At the time of injection-molding of the probe lens 31, the injection material may be inserted in a state when the case 30 is inserted at the mold. The mold may be closed after inserting the injection material of the probe lens 31, and predetermined heat and pressure may be applied. At this time, by vacuuming and defoaming an inside the mold, the injection material may be evenly introduced into an inside the groove 301. After the heat and pressure are applied for a predetermined period of time, the mold is cooled, and then the module of which the case 30 is coupled to the probe lens 31 may be separated from the mold.

As described above, as the probe lens 31 is injection-molded while the case 30 is inserted in a mold for injection molding, the probe lens 31 may be formed to correspond to the shape of the groove 301 formed at the rear surface of the case 30. From the above, a method of the probe lens 31 injection-molded as the case 30 is inserted in the mold is described, while a method of probe lens 31 coupled to the rear surface 30r of the case 30 is not limited to the description provided above.

The shape of the groove 301 may be provided in various forms. As illustrated in FIG. 5A, the groove 301 may be provided to form a rectangular cross section at the inner side surface of the case 30, or, as illustrated on FIG. 5D, the groove 301 may be provided to form a rhombus-shaped cross section at the inner side surface of the case 30. As illustrated on FIG. 5B, the groove 301 may be provided to form a trapezoidal cross section at the inner side surface of the case 30. As illustrated on FIG. 5C, the groove 301 may be provided to form a triangular cross section at the inner side surface of the case 30. In addition, as illustrated on FIG. 5E, the groove 301 may be provided to form a semicircular cross section at the inner side surface of the case 30.

However, the shape of the groove 301 is not limited to the descriptions provided above.

In the exemplary embodiment, the groove 301 may be formed at the rear surface 30r/ inner side surface of the case 30, and the probe lens 31 positioned at a rear side of the case 30 may be formed to have a shape to correspond to the shape of the groove 301.

In an exemplary embodiment, ultrasonic gel G is applied at a body B to perform an ultrasonic test. The ultrasonic probe 3 may obtain ultrasonic images with respect to the body B while making contact with the body B applied with the ultrasonic gel G. The ultrasonic probe 3 may be exposed to the ultrasonic gel G as the ultrasonic probe 3 is provided to capture ultrasonic images while making contact with the body B applied with the ultrasonic gel G as illustrated in FIG. 4. The ultrasonic gel G remaining at the ultrasonic probe 3 may be removed by cleansing solution. Therefore, the ultrasonic probe 3 may be exposed to the ultrasonic gel G when performing ultrasonic imaging, and may also be exposed to the cleansing solution provided to remove the ultrasonic gel G.

In the related art, with respect to the front of an ultrasonic probe 3 at which a probe lens is positioned, when frequently exposed to ultrasonic gel G or cleansing solution, the ultrasonic gel G or the cleansing solution is introduced into an area between of the probe lens and the case. The ultrasonic gel G or the cleansing solution may weaken the coupling force between the probe lens and the case, and may separate the probe lens and the case. In addition, the ultrasonic gel G or the cleansing solution introduced into the area between of the probe lens and the case may cause corrosion on components provided inside the ultrasonic probe, such as the transducer and a printed circuit board.

In the related art, as to improve adhesiveness between the probe lens and the case, a method of using an adhesive promoter in the area between the probe lend and the case or a method of coupling the probe lens by increasing coarseness of the rear surface 30r of the case by means of sanding is used. However, even in the use of the method of using the adhesive promoter or the sanding process, a prevention of the introduction of the ultrasonic gel G or the cleansing solution through the area between of the probe lens and the case is found to be difficult. Particularly, as the area of the probe lens making contact with the body B is increased as to capture ultrasonic images, more incidences of the ultrasonic gel G or the cleansing solution introduced into the area between the case and the probe lens frequently occur.

In the exemplary embodiment, as the groove 301 is formed at the rear surface 30r/ inner side surface of the case 30 and as the probe lens 31 is injection-molded to correspond to the shape of the groove 301, the area at which the case 30 and the probe lens 31 are coupled to may be increased. Therefore, the case 30 and the probe lens 31 may be coupled by stronger adhesive force than the method of which the case 30 and the probe lens 31 are coupled conventionally. As the case 30 and the probe lens 31 are coupled by stronger adhesive force, a phenomenon of which the case 30 and the probe lens 31 are separated by the ultrasonic gel G or the cleansing solution may be prevented.

In addition, as the shape of the groove 301 is provided at the coupling potion at which the case 30 and the probe lens 31 are coupled, the path of the ultrasonic gel G or the cleansing solution introduced to an inside the ultrasonic probe 3 through the area between of the probe lens 31 and the case 30 is lengthened. Even when the ultrasonic gel G or the cleansing solution is introduced through the area between of the probe lens 31 and the case 30, the ultrasonic gel G or the cleansing solution is introduced along the surface forming the groove 301, and thus, when compared with the conventional case, the path for the ultrasonic gel G or the cleansing solution to reach the transducer 32 or the printed circuit board positioned at the inside the ultrasonic probe 3 may be lengthened. Therefore, corrosion of the inside components of the ultrasonic probe 3 by chemical products such as the ultrasonic gel G and the cleansing solution may be effectively prevented.

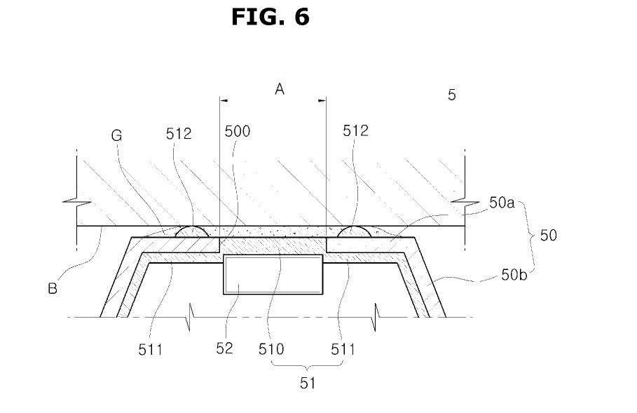

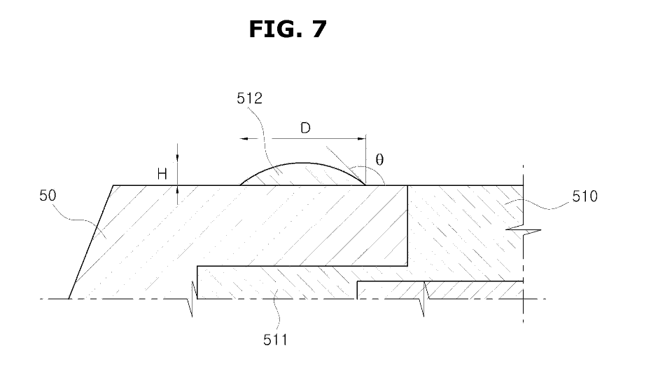

FIG. 6 is a cross-sectional view illustrating an ultrasonic probe 5 in accordance with an exemplary embodiment, and FIG. 7 is a drawing illustrating a portion of an ultrasonic probe 5 in accordance with an exemplary embodiment.

Referring to FIG. 6 and FIG. 7, an ultrasonic probe 5 in accordance with an exemplary embodiment may be provided with a protrusion 512. A case 50 may include a first case portion 50a at which an opening 500 is formed, and a second case portion 50b bent at and extended from the first case portion 50a. The first case portion 50a may include a front surface 50f of the case 50, and the second case portion 50b may include a side surface of the case 50. The protrusion 512 may protrude from the front surface 50f of the first case portion 50a. A probe lens 51 may include a first portion 510 exposed through the opening 500, and a second portion 511 coupled to an inner side surface of the case 50.

The protrusion 512 may be injection-molded integrally with the probe lens 51 or the case 50. For example, the protrusion 512 may be injection-molded integrally with the probe lens 51. At this time, when the probe lens 51 is injection-molded as the case 50 is inserted in a mold, the protrusion 512 may be formed by the shape of the mold. A hole (not shown) may be formed at the first case portion 50a of the case 50, and the injection material may be injected into a space at an inside the mold corresponding to the protrusion 512.

The protrusion 512 may be provided to make contact with the body B while protruding from a front surface 50f of the ultrasonic probe 5 as shown in FIG. 6. At the time of capturing ultrasonic images, by having the protrusion 512 make contact with the body B while the protrusion 512 is protruded toward the ultrasonic probe 5, repetitive frictions between the probe lens 51 and the body B and the pressure applied at the probe lens 51 may be prevented.

The probe lens 51 may be injection-molded with softer material when compared to the case 50, and pressure may be applied at the probe lens 51 by the force that presses the body B at the time of capturing ultrasonic images, and friction may be applied at a front surface of the probe lens 51 as the probe lens 51 is provided to capture ultrasonic images while moving at the surface of the body B. Irregular abrasion may be generated at the probe lens 51 by the pressure and the friction as such, and the regularity of the captured ultrasonic images may be reduced by the abrasion as such. In addition, the ultrasonic gel G or the cleansing solution may be introduced to an inside the ultrasonic probe 5 along an inner side surface of the case 50 that is coupled to a portion of the abrasion of the probe lens 51. The transducer 52 or the printed circuit board positioned at an inside the ultrasonic probe 5 may be damaged by the ultrasonic gel G or the cleansing solution introduced to the inside the ultrasonic probe 5.

In the exemplary embodiment, as the protrusion 512 protruding from a front surface 50f of the ultrasonic probe 5 is provided by means of reducing the pressure and friction applied at the probe lens 51, the abrasion of the probe lens 51 is prevented, and the structure at the inside the ultrasonic probe 5 may be prevented from being damaged. In addition, the ultrasonic gel G provided in between the body B and the ultrasonic probe 5 may improve lubricativeness as the ultrasonic gel G is provided to remain well by the protrusion 512 at a capturing area A of ultrasonic images.

The protrusion 512 may be positioned at an outer side of the capturing area A such that the capturing area A is not interfered. The height H of the protrusion 512, the angle θ that is formed by the protrusion 512 and the first case portion 50a of the case 50 at which the protrusion 512 is positioned, and the width D of the protrusion 512 may vary according to the shape and size of the ultrasonic probe 5.

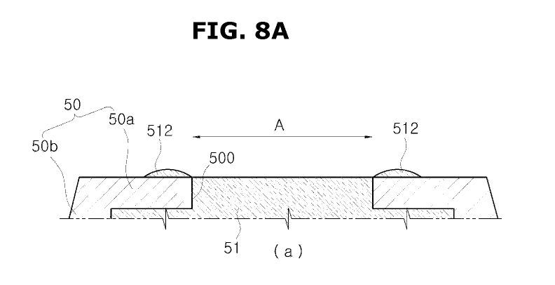

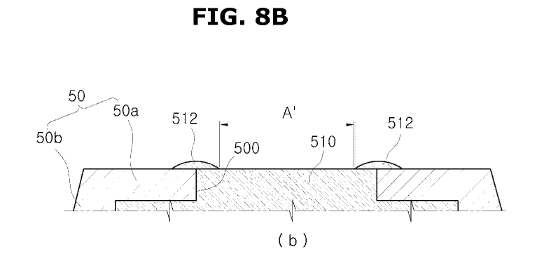

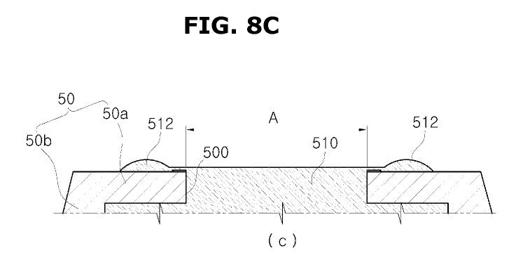

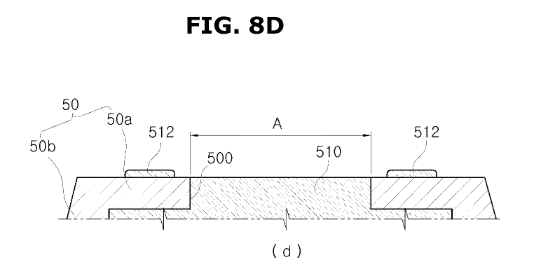

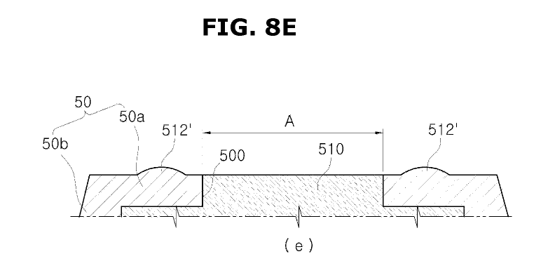

FIG. 8A to FIG. 8E are drawings illustrating various examples with respect to a shape of an inner side surface of an ultrasonic probe 5 in accordance with another embodiment of the present disclosure.

As illustrated from FIG. 8A to FIG. 8E, the ultrasonic probe 5 in accordance with an exemplary embodiment may be provided with various shapes of the protrusion 512. As illustrated on FIG. 8A, the protrusion 512 may be injection-molded integrally with the case 50 as to be adjacent to the capturing area A formed by the opening 500 of the case 50.

As illustrated on FIG. 8B, at least a portion of the protrusion 512 may be provided to cover a portion of an inner side of the opening 500 of the case 50 at which the probe lens 51 is exposed. At this time, a distortion may be generated at an image captured by ultrasonic signals passing at the portion of the protrusion 512, and thus, an actual capturing area A′ may be the portion positioned at an inner side of the protrusion 512.

As illustrated on FIG. 8C, the protrusion 512 may be provided such that the probe lens 51 connected to the protrusion 512 is provided to cover a portion of an upper surface of the first case portion 50a of the case 50. At this time, the capturing area A may be a portion that corresponds to the area of the opening 500.

As illustrated on FIG. 8D, the protrusion 512 may be provided in the shape of a semi-sphere, or may be provided in the shape of a polyhedron.

A protrusion 512′ may be injection-molded integrally with the case 50. As illustrated on FIG. 8E, the protrusion 512′ may be injection-molded with identical material of the case 50, or the probe lens 51 may be injection-molded as the case 50 is inserted in a mold.

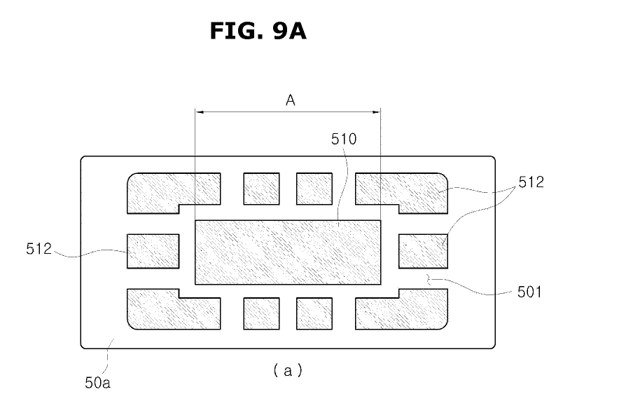

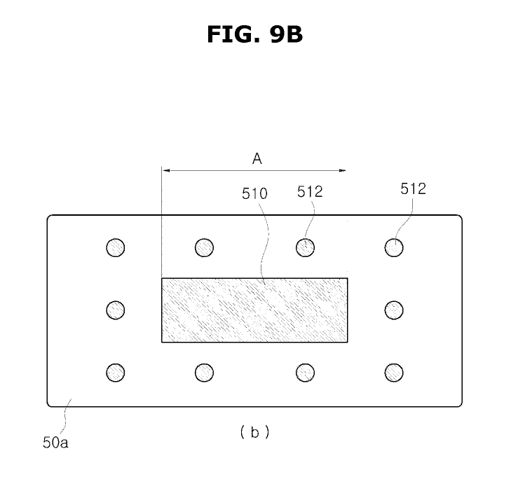

FIG. 9A and FIG. 9B are drawings illustrating a surface of the ultrasonic probe 5 in accordance with an exemplary embodiment.

Referring to FIG. 9A and FIG. 9B, the protrusion 512 in accordance with an exemplary embodiment may be provided in a plurality of protrusions thereof while spaced apart to each other by a predetermined distance at a front surface 50f of the ultrasonic probe 5. In a case when the plurality of protrusions 512 is provided while the protrusions are spaced apart to one another, the ultrasonic gel G applied at a portion at which the capturing area A is positioned may be moved toward an outer side of the capturing area A through a moving path 501 provided between adjacent protrusions 512 of the plurality of protrusions. Therefore, when the ultrasonic probe 5 is moved along a surface of the body B, the lubrication may be improved by the ultrasonic gel G.

As illustrated in FIG. 9A and FIG. 9B, the area occupied by the each protrusion 512 of the plurality of protrusions 512, which is provided at the front surface 50f of the ultrasonic probe 5, at the front surface 50f of the ultrasonic probe 5 may be varied.

As the above, an abrasion of the probe lens may be prevented as the protrusion is provided at a front surface 50f of the ultrasonic probe 5, and a damage of the transducer or the printed circuit board at an inside the ultrasonic probe by the introduction of the ultrasonic gel or the cleansing solution to an inside the ultrasonic probe may be prevented.

While exemplary embodiments have been particularly shown and described, it would be appreciated by those skilled in the art that various changes may be made therein without departing from the principles and spirit of the inventive concept, the scope of which is defined in the following claims.

What is claimed is:

1. An ultrasonic probe, comprising:
a case comprising:
an opening; and
a coupling portion; and
a probe lens comprising:
a first portion being exposed through the opening and a second portion coupled to the coupling portion of the case,
wherein one of the coupling portion of the case and the second portion of the probe lens comprises a groove, and
other one of the coupling portion of the case and the second portion of the probe lens comprises a shape matching the groove to couple the case and the probe lens to each other.

2. The ultrasonic probe of claim 1, wherein the case comprises a protrusion which protrudes from a front surface of the case.

3. The ultrasonic probe of claim 2, wherein the protrusion is integrally formed with the probe lens.

4. The ultrasonic probe of claim 2, wherein the protrusion is integrally formed with the case.

5. The ultrasonic probe of claim 2, wherein the protrusion is provided at an outer side of the case with respect to the opening.

6. The ultrasonic probe of claim 2, wherein the protrusion is one of a plurality of protrusions, which are spaced apart with respect to one another.

7. The ultrasonic probe of claim 2, wherein the protrusion comprises a cross-section in a shape of a semi-sphere.

8. The ultrasonic probe of claim 2, wherein the protrusion comprises a cross-section in a shape of a polyhedron.

9. The ultrasonic probe of claim 2, wherein the probe lens comprises an injection-molded lens.

10. The ultrasonic probe of claim 1, wherein the case comprises:
a first case portion positioned at a front side of the ultrasonic probe; and
a second case portion bent at and extended from the first case portion, and
wherein the groove is formed at an inner side surface of the first case portion or at an inner side surface of the second case portion.

11. The ultrasonic probe of claim 10, wherein the second portion extends from the first portion and is coupled to the inner side surface of at least one of the first case portion and the second case portion.

12. The ultrasonic probe of claim 10, wherein a first surface of the first case portion comprises a plurality of protrusions.

13. The ultrasonic probe of claim 1, wherein a shape of the groove corresponds to a shape of a mold used for injection molding of the case.

14. The ultrasonic probe of claim 1, wherein:
the groove comprises a carved groove.

15. An ultrasonic probe comprising:
a case having an opening; and
a probe lens exposed through the opening, wherein:
the case comprises grooves provided on a portion of a rear surface of the case, and the probe lens comprises a shape corresponding to the grooves and is configured to be mounted at the rear surface of the case, and
the case further comprises protrusions provided at a front surface of the case and configured to make contact with an object.

16. The ultrasonic probe of claim 15, wherein the groove is provided over an entire area of the rear surface of the case coupled to the probe lens.

17. The ultrasonic probe of claim 15, wherein the protrusion is integrally formed with the probe lens.

18. The ultrasonic probe of claim 17, wherein the protrusion is provided at an outer side of the case with respect to the opening and is formed to be spaced apart from the opening.

19. The ultrasonic probe of claim 15, wherein a moving path is provided between adjacent protrusions of the protrusions and configured to allow ultrasonic gel to move through the moving path.

20. The ultrasonic probe of claim 15, wherein the protrusion is integrally formed with the case.
